(19) United States
(12) Patent Application Publication
(10) Pub. No.: US 2009/0233947 A1
(43) Pub. Date: Sep. 17, 2009

(54) 2,3-Substituted fused bicyclic pyrimidin-4(3H)-ones which modulating the function of the vanilloid -1 receptor (VR1)

(76) Inventors: Tracy Bayliss, Walthamstow (GB); Rebecca Elizabeth Brown, Cambridge (GB); Gregory John HollingWorth, Brentwood (GB); A. Brian Jones, Needham, MA (US)

Correspondence Address: MERCK AND CO., INC, P O BOX 2000, RAHWAY, NJ, 07065-0907, US

(21) Appl. No.: 11/920,065

(22) PCT Filed: May 10, 2006
(86) PCT No.: PCT/GB2006/050100

§ 371 Date: Jan. 14, 2009

(30) Foreign Application Priority Data
May 11, 2005 (GB) 0509573.2

(51) Int. Cl. A61K 31/522; C07D 473/30; A61P 25/00

(52) U.S. Cl. 514/263.23; 544/265; 514/263.3; 544/276

(57) ABSTRACT

Compounds of formula (I) which are useful as therapeutic compounds, particularly in the treatment of pain and other conditions ameliorated by the modulation of the function of the vanilloid-1 receptor (VR1, also known as TRPV1).

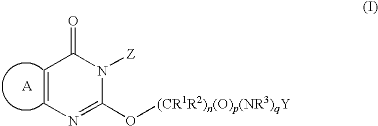

The present invention is concerned with 2,3-substituted fused bicyclic pyrimidin-4(3H)-ones and analogues and derivatives thereof as well as pharmaceutically acceptable salts and prodrugs thereof, which are useful as therapeutic compounds, particularly in the treatment of pain and other conditions ameliorated by the modulation of the function of the vanilloid-1 receptor (VR1, also known as TRPV1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly the VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)—VR1 IC₅₀ of 420 nM. Other sub-micromolar antagonists has also been reported recently (Lee et al, Bioorg. Med. Chem., 9:1713, 2001; Park et al, Bioorg. Med. Chem. Lett., 13:601, 2003; Yoon et al, Bioorg. Med. Chem. Lett., 13:1549, 2003; Lee et al, Bioorg. Med. Chem., 12:3411, 2004; McDonnell et al, Bioorg. Med. Chem. Lett., 14:531, 2004; Ryu et al, Bioorg. Med. Chem. Lett., 14:1751, 2004; Rami et al, Bioorg. Med. Chem. Lett., 14:3631, 2004; Gunthorpe et al, Neuropharmacology 46:133, 2004; Doherty et al, J. Med. Chem., 48:71, 2005), but these reports provide no evidence for in vivo efficacy. A high affinity antagonist has been derived from the potent agonist resiniferatoxin; iodo-resiniferatoxin (Wahl et al., Mol. Pharmacol., 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002).

EP-A-0807633 (Pfizer Inc.) discloses structurally related AMPA receptor antagonists for treating neurodegenerative and CNS-trauma related conditions.

WO-A-9733890 (Novartis AG) discloses structurally related compounds as pesticides.

The compounds of the present invention have advantageous properties, such as good in vivo efficacy.

The compounds of the present invention unexpectedly show improved pharmacokinetic properties, such as improved metabolic stability.

We herein describe another novel series of VR1 modulators. These comprise predominantly VR1 antagonists but encompass VR1 partial antagonists and VR1 partial agonists. Such compounds have been shown to be efficacious in animal models of pain.

The present invention provides a compound of formula I:

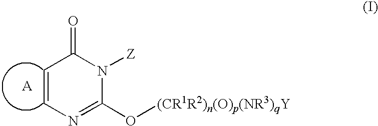

wherein:

A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;

A is optionally substituted by one, two or three groups independently chosen from halogen, hydroxy, S(O)ᵣC₁₋₄alkyl, S(O)NR⁴R⁵, —NR^{X}S(O)ᵣC₁₋₄alkyl, formyl, C₁₋₄alkylcarbonyl, C₁₋₄alkyl, haloC₁₋₆allyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₄alkylamino, di(C₁₋₆alkyl)amino, aminoC₁₋₆alkyl, aminoC₁₋₆alkoxy, C₁₋₆alkylaminoC₁₋₆alkyl, di(C₁₋₆alkyl)aminoC₁₋₆alkyl, C₁₋₆alkylaminoC₁₋₆alkoxy, di(C₁₋₆alkyl)aminoC₁₋₆alkoxy, and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, N 5 as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

R¹ and R² are independently hydrogen, hydroxy, halogen or C₁₋₆alkyl;

R³ is hydrogen or C₁₋₆alkyl;

n is zero, one, two, three or four;

p is zero or one;

when p is zero, q is zero or one;

when p is one, q is zero;

r is zero, one or two;

Y is hydrogen, C₁₋₆alkoxy, haloC₁₋₆alkoxy, C₁₋₆alkyl, haloC₁₋₆alkyl, hydroxyC₁₋₆alkyl, aminoC₁₋₆alkyl, carboxyC₁₋₆allyl, C₃₋₇cycloalkyl, haloC₃₋₇cycloalkyl; or a phenyl ring, a five-membered heteroaromatic ring containing one, two, three or four heteroatoms independently chosen from O, N and S, providing that no more than one O or S atom is present, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, a six-membered saturated ring containing one or two heteroatoms independently chosen from O and N, the ring being optionally substituted by one or more groups independently selected from halogen, C₁₋₆alkyl, C₂₋₆alkenyl, C₂₋₄alkynyl, nitro, cyano, C₃₋₇cycloalkyl, hydroxy, C₁₋₄alkoxy, haloC₁₋₄alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;

each R⁴ and R⁵ is independently hydrogen or C₁₋₆alkyl or R⁴ and R⁵, together with the nitrogen atom to which they are attached, may form a saturated 4-7 membered ring;

R^{X} is hydrogen or C₁₋₆alkyl;

Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by halogen, hydroxy, cyano, nitro, NR⁴R⁵ as defined above, S(O)ᵣNR⁴R⁵, —NR^{X}S(O)ᵣC₁₋₄alkyl, S(O)ᵣC₁₋₄alkyl, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;

provided that when n is zero then p and q are both zero and Y is not hydrogen, C₁₋₆alkoxy or haloC₁₋₆alkoxy;

provided that when A is thiophene, n is one, p and q are zero, R¹ and R² are hydrogen and Z is 4-chlorophenyl then Y is not 3-fluorophenyl;

or a pharmaceutically acceptable salt or tautomer thereof.

A is preferably a fused five-membered heteroaromatic ring containing 1, 2 or 3 heteroatoms independently chosen from O, N and S, providing that no more than one O or S atom is present.

In one embodiment A is not a fused thiophene.

More preferably, A is a fused five-membered heteroaromatic ring containing 1, 2, or 3 heteroatoms independently chosen from O and N, providing that no more than one 0 is present.

In one embodiment A is a fused pyridine, thiazole or imidazole. More particularly, A is a fused thiazole or imidazole. Favourably A is a fused imidazole.

A is preferably unsubstituted or substituted by halogen, hydroxy, C₃₋₆cycloalkyl, C₁₋₄alkyl, haloC₁₋₄alkyl, C₁₋₄alkoxy, haloC₁₋₄alkoxy or phenyl. A further preferred substituent is hydroxyC₁₋₄alkyl, aminoC₁₋₄alkyl, C₁₋₄alkylaminoC₁₋₄alkyl or di(C₁₋₄alkyl)aminoC₁₋₄alkyl.

A is preferably unsubstituted or substituted by halogen, hydroxy, C₃₋₅cycloalkyl, C₁₋₄alkyl, haloC₁₋₄allyl, C₁₋₄alkoxy or haloC₁₋₄alkoxy. More preferably A is unsubstituted or substituted by C₁₋₄alkyl, C₃₋₅cycloalkyl or haloC₁₋₄alkyl. Favourably A is unsubstituted or substituted by methyl, ethyl, trifluoroethyl or cyclopropyl. More particularly, A is unsubstituted or substituted by methyl, ethyl, 2,2,2-trifluoroethyl or cyclopropyl.

Preferably A is unsubstituted or substituted by one or two groups. More particularly A is monosubstituted.

In one embodiment A is monosubstituted by methyl or ethyl, such as methyl.

Where A is substituted by hydroxy group tautomerism may occur. For example, when A is fused imidazole, tautomerism may occur to form an imidazolone.

R¹ and R² are independently preferably hydrogen or C₁₋₄alkyl. Most particularly R¹ and R² are both hydrogen.

R³ is preferably hydrogen or C₁₋₂alkyl. R³ may be hydrogen.

n is preferably one, two or three. In one embodiment n is two.

p is preferably zero.

In one embodiment p and q are both zero.

In one embodiment Y is hydrogen, C₁₋₄ alkyl, haloC₁₋₆alkyl, C₃₋₇cycloalkyl; or a phenyl ring, a five-membered heteroaromatic ring containing one, two, three or four heteroatoms independently chosen from O, N or S, at most one heteroatom being O or S, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, the ring being optionally substituted by one or more groups independently chosen from halogen, C₁₋₄alkyl, hydroxy, C₁alkoxy, haloC₁₋₄alkyl, phenyl, haloC₁₋₄alkoxy and NR⁴R⁵ where R⁴ and R⁵ are independently C₁₋₄alkyl or, R⁴ and R⁵, together with the nitrogen atom to which they are attached, form a 5-6 membered saturated ring.

Y is preferably hydrogen, haloC₁₋₄alkyl, a phenyl ring or a five-membered heteroaromatic ring containing one, two, three or four heteroatoms independently chosen from O, N and S, providing that no more than one O or S atom is present, the ring being optionally substituted by one or more groups independently selected from halogen, C₁₋₄alkyl and haloC₁₋₄alkyl.

More particularly, Y is hydrogen, haloC₁₋₄alkyl, a phenyl ring or a thiazole ring, the ring being optionally substituted by haloC₁₋₄alkyl.

Favourable Y groups are hydrogen, trifluoromethyl, trifluoromethylphenyl and trifluoromethylthiazole. A further preferred Y group is methyl.

In one embodiment Y is trifluoromethyl.

Thus, specific Y groups are hydrogen, trifluoromethyl, 4-trifluoromethylphenyl, 2-(trifluoromethyl)-1,3-thiazol-4-yl and 4-(trifluoromethyl) 1,3-thiazol-2-yl.

In one embodiment Y is not phenyl.

In another embodiment Y is not hydrogen.

Z is preferably an optionally substituted phenyl or pyridinyl. More particularly Z is an optionally substituted phenyl.

Z is preferably unsubstituted or substituted by one or two substituents chosen from cyano, halogen, C₁₋₄alkyl, haloC₁₋₄alkyl, C₁₋₄alkoxy, haloC₁₋₄alkoxy, amino, C₁₋₄alkylamino and di(C₁₋₄alkyl)amino. Particular substituents include chlorine, trifluoromethyl, cyano, methyl, fluorine, ethoxy, trifluoromethoxy, bromine, dimethylamino, methoxy and isopropoxy. Favoured substituents are cyano and halogen. Particular substituents include cyano, fluorine and chlorine.

Preferably, Z is monosubstituted.

Thus, specific Z groups are 4-chlorophenyl, 4-fluorophenyl and 4-cyanophenyl.

The present invention also provides compounds of formula IA:

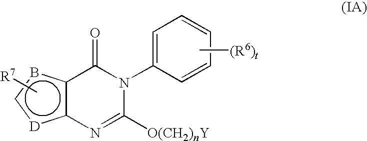

wherein:

n and Y are as defined above;

t is zero, one or two;

one of B and D is N and the other N or S;

R⁶ is cyano, halogen, C₁₋₄alkyl, haloC₁₋₄alkyl, C₁₋₄alkoxy, haloC₁₋₄alkoxy, amino, C₁₋₄alkylamino or di(C₁₋₆)alkylamino; and

R⁷ is hydrogen, halogen, hydroxy, C₃₋₅cycloalkyl, C₁₋₄allyl, haloC₁₋₄alkyl, C₁₋₄alkoxy or haloC₁₋₄alkoxy;

provided that when n is zero then Y is not hydrogen, C₁₋₆alkoxy or haloC₁₋₆alkoxy;
or a pharmaceutically acceptable salt or tautomer thereof.

The favoured identities with reference to formula IA are as defined previously for formula I mutatis mutandis.

Preferably, t is one or two. In one embodiment t is one.

In one embodiment B and D are both N.

In another embodiment B is N and D is S.

In one embodiment R⁶ is substituted at the para position.

Preferably, each R⁶ is independently cyano or halogen. More particularly R⁶ is independently fluorine, chlorine or cyano.

Preferably, R⁷ is C₁₋₄alkyl, C₃₋₅cycloalkyl or haloC₁₋₄alkyl. More particularly R⁷ is methyl, ethyl, trifluoroethyl or cyclopropyl.

In one embodiment, when B and D are both N then R⁷ is attached at D.

When one of B and D is N and the other S then R⁷ is attached at the carbon ring atom.

The present invention also provides compounds of formula IB:

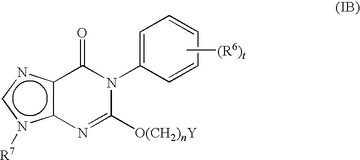

wherein:

n, t, R⁶, R⁷ and Y are as defined above;

or a pharmaceutically acceptable salt or tautomer thereof.

The favoured identities with reference to formula IB are as defined previously for formula IA mutatis mutandis.

Particular embodiments of the invention include:
1-(4-chlorophenyl)-9-cyclopropyl-2-methoxy-1,9-dihydro-6H-purin-6-one;
4-[9-methyl-6-oxo-2-(3,3,3-trifluoropropoxy)-6,9-dihydro-1H-purin-1-yl]benzonitrile;
1-(4-chlorophenyl)-9-ethyl-2-(3,3,3-trifluoropropoxy)-1,9-dihydro-6H-purin-6-one;
1-(4-chlorophenyl)-9-methyl-2-{[2-(trifluoromethyl)-1,3-thiazol-4-yl]methoxy}-1,9-dihydro-6H-purin-6-one;
1-(4-chlorophenyl)-9-methyl-2-{[4-(trifluoromethyl)-1,3-thiazol-2-yl]methoxy}-1,9-dihydro-6H-purin-6-one;
1-(4-fluorophenyl)-9-methyl-2-(3,3,3-trifluoropropoxy)-1,9-dihydro-6H-purin-6-one;
1-(4-chlorophenyl)-9-methyl-2-(3,3,3-trifluoropropoxy)-1,9-dihydro-6H-purin-6-one;
1-(4-chlorophenyl)-9-methyl-2-{2-[4-(trifluoromethyl)phenyl]ethoxy}-1,9-dihydro-6H-purin-6-one;
1-(4-chlorophenyl)-9-(2,2,2-trifluoroethyl)-2-(3,3,3-trifluoropropoxy)-1,9-dihydro-6H-purin-6-one;
1-(4-fluorophenyl)-9-methyl-2-(4,4,4-trifluorobutoxy)-1,9-dihydro-6H-purin-6-one;
1-(4-chlorophenyl)-9-methyl-2-(4,4,4-trifluorobutoxy)-1,9-dihydro-6H-purin-6-one;
1-(4-fluorophenyl)-9-methyl-2-{[4-(trifluoromethyl)-1,3-thiazol-2-yl]methoxy}-1,9-dihydro-6H-purin-6-one;
1-(4-fluorophenyl)-9-methyl-2-{[2-(trifluoromethyl)-1,3-thiazol-4-yl]methoxy}-1,9-dihydro-6H-purin-6-one;
   and pharmaceutically acceptable salts and tautomers thereof.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy.

As used herein, the terms “haloC₁₋₆alkyl” and “haloC₁₋₄alkoxy” means a C₁₋₄alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂F, CHF₂, CH₂CH₂F, CH₂CHF₂, CH₂CF₃, OCF₃, OCH₂CH₂F, OCH₂CHF₂ or OCH₂CF₃, and most especially CF₃ and OCF₃.

The cycloalkyl groups referred to herein may represent, for example, cyclopropyl, cyclobutyl, cyclopentyl or cyclohexyl. Such groups also include, for example, cyclopropylmethyl and cyclohexylmethyl.

As used herein, the terms “alkenyl” and “alkynyl” as a group or part of a group means that the group is straight or branched. Examples of suitable alkenyl groups include vinyl and allyl. A suitable alkynyl group is acetylene or propargyl.

When used herein, the term “halogen” means fluorine, chlorine, bromine and iodine. The most preferred halogens are fluorine and chlorine, especially chlorine.

Examples of 6-membered saturated rings are morpholine, piperidine and piperazine.

Examples of 6-membered heteroaromatic rings are pyridine, pyrimidine, pyrazine, pyridazine and triazine.

Examples of 5-membered heteroaromatic rings are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

Examples of 9- or 10-membered fused bicyclic heteroaromatic rings include benzofuran, benzothiophene, benzimidazole, benzoxazole, benzothiazole, benzisothiazole, quinoline, isoquinoline and cinnoline.

In a further aspect of the present invention, the compounds of formula I may be prepared in the form of a pharmaceutically acceptable salt, especially an acid addition salt.

For use in medicine, the salts of the compounds of formula I will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. A further salt is the acid addition salt with benzenesulfonic acid. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula I with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula I above. In general, such N-oxides may be formed on any available nitrogen atom. The N-oxides may be formed by conventional means, such as reacting the compound of formula I with Oxone® in the presence of wet alumina.

The present invention includes within its scope prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula I may also exist in tautomeric forms and the invention includes within its scope both mixtures and separate individual tautomers.

The compounds may exist in different isomeric forms, all of which are encompassed by the present invention.

The present invention further provides pharmaceutical compositions comprising one or more compounds of formula I in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula I required for use in any treatment will vary not only with the particular compounds or composition selected but also with the route of administration, the nature of the condition being treated, and the age and condition of the patient, and will ultimately be at the discretion of the attendant physician.

The invention further provides a compound of formula I as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, for example stump pain and phantom limb pain, peripheral neuropathies, tic douloureux, atypical facial pain, nerve root damage, and arachnoiditis; itching conditions including pruritis, itch due to hemodialysis, contact dermatitis, vulvar vestibulitis, insect bites and skin allergies; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy, post-herpetic neuralgia, causalgia (reflex sympathetic dystrophy—RSD, secondary to injury of a peripheral nerve), neuritis (including sciatic neuritis, peripheral neuritis, polyneuritis, optic neuritis, postfebrile neuritis, migrating neuritis, segmental neuritis and Gombault's neuritis, neuronitis, neuralgias (including cervicobrachial neuralgia, cranial neuralgia, geniculate neuralgia, glossopharynigial neuralgia, migranous neuralgia, idiopathic neuralgia, intercostals neuralgia, mammary neuralgia, mandibular joint neuralgia, Morton's neuralgia, nasociliary neuralgia, occipital neuralgia, red neuralgia, Sluder's neuralgia, splenopalatine neuralgia, supraorbital neuralgia and vidian neuralgia), AIDS-related neuropathy and MS related neuropathy; “non-painful” neuropathies; oral neuropathic pain; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence (including overflow incontinence, urge incontinence and stress incontinence) including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis and cough; autoimmune diseases; immunodeficiency disorders; hot flushes; postmuastectomy pain syndrome; dental pain, such as toothache and denture pain; reflex sympathetic dystrophy; trigeminal neuralgia; fibromyalgia; Guillain-Barre syndrome; meralgia paresthetica; burning-mouth syndrome; Charcot's pains; intestinal gas pains; menstrual pain; hemorrhoidal pain; dyspeptic pains; angina; homotopic and heterotopic pain; pain associated with venom exposure; other trauma associated pain such as pain from cuts, bruises, broken bones and burns); and carpel tunnel syndrome. The compounds of the present invention may also be used to treat depression, hiccups and obesity. They may also be used to treat gastro-oesophageal reflux disease (GERD), particularly the pain associated with GERD.

Thus, according to a further aspect, the present invention provides a compound of formula I for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula I or a composition comprising a compound of formula I.

According to a further or alternative aspect, the present invention provides a compound of formula I for use in the manufacture of a medicament for the treatment or prevention of a disease or condition in which pain and/or inflammation predominates.

The present invention also provides a method for the treatment or prevention of a disease or condition in which pain and/or inflammation predominates, which method comprises administration to a patient in need thereof of an effective amount of a compound of formula I or a composition comprising a compound of formula I.

According to a further aspect of the present invention, it may be desirable to treat any of the aforementioned conditions with a combination of a compound according to the present invention and one or more other pharmacologically active agents suitable for the treatment of the specific condition. The compound of formula I and the other pharmacologically active agent(s) may be administered to a patient simultaneously, sequentially or in combination.

Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR²B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin, asthma treatments (such as υ₂-adrenergic receptor agonists or leukotriene D₄ antagonists (e.g. montelukast), gold compounds, corticosteroids, methotrexate, tumor necrosis (TNF) receptor antagonists, anti-TNF alpha antibodies, anti-C5 antibodies and interluekin-1(IL-1) receptor antagonists.

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib, tilicoxib, flurbiprofen, naproxen sodium, combinations of diclofenac sodium and misoprostol, oxaprozin, diflunisal, fenoprofen, calcium, sodium nabumetone, sulfasalazine, tolmetin sodium, hydroxychloroquine, acetylsalicylic acid, sodium salicylate, choline and magnesium salicylates, salsalate, cortisone, dexamethasone, methylprednisolone, prednisolone, prednisolone sodium phosphate, prednisone and lumiracoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene, pentazocine, alphaprodine, anileridine, bezitramide, diacetyldihydromorphine, diphenoxylate, ethylmorphine, heroin, isomethadone, levomethorphan, levorphanol, metazocine, methorphan, metopon, opium extracts, opium fluid extracts, powdered opium, granulated opium, raw opium, tincture of opium, oxycodone, paregoric, pethidine, phenazocine, piminodine, racemethorphan, racemorphan, thebaine, acetorphine, acetyldihydrocodeine, acetylmethadol, allylprodine, alphracetylmethadol, alphameprodine, alphamethadol, benzethidine, benzylmorphine, betacetylmethadol, betameprodine, betamethadol, betaprodine, clonitazene, codeine methylbromide, codeine-N-oxide, cyprenorphine, desomorphine, dextromoramide, diampromide, diethylthiambutene, dihydromorphine, dimenoxadol, dimepheptanol, dimethylthiamubutene, dioxaphetyl, butyrate, dipipanone, drotebanol, ethanol, ethylmethylthiambutene, etonitazene, etorphine, etoxeridine, furethidine, hydromorphinol, hydroxypethidine, ketobemidone, levomoramide, levophenacylmorphan, methyldesorphine, methyldihydromorphine, morpheridine, morphine methylpromide, morphine methylsulfonate, morphine-N-oxide, myrophin, naloxone, nalbuyphine, naltyhexone, nicocodeine, nicomorphine, noracymethadol, norlevorphanol, normethadone, normorphine, norpipanone, pentazocaine, phenadoxone, phenampromide, phenomorphan, phenoperidine, piritramde, pholcodine, proheptazoine, properidine, propiran, racemoramide, thebacon and trimeperidine; or a pharmaceutically acceptable salt thereof. Suitable anti-migraine agents of use in conjunction with a compound of the present invention include CGRP-antagonists, ergotamines or 5-HT, agonists, especially sumatriptan, naratriptan; zolmatriptan or rizatriptan.

Thus, for example, for the treatment or prevention of cough, a compound of the present invention may be used in conjunction with other medication designed to treat this condition, such as antibiotics, anti-inflammatory agents, cystinyl leukotrienes, histamine antagonists, corticosteroids, opioids, NMDA antagonists, proton pump inhibitors, nociceptin, neurokinin (NK1, NK2 and NK3) and bradykinin (Bk1 and Bk2) receptor antagonists, cannabinoids, blockers of Na+-dependent channels and large conductance Ca(2⁺)dependent K+-channel activators. Specific agents include dexbrompheniramine plus pseudoephedrine, loratadine, oxymetazoline, ipratropium, albuterol, beclomethasone, morphine, codeine, pholcodeine and dextromethorphan.

Thus, for example, for the treatment or prevention of urinary incontinence, a compound of the present invention may be used in conjunction with other medication designed to treat this condition, such as estrogen replacement therapy, progesterone congeners, electrical stimulation, calcium channel blockers, antispasmodic agents, cholinergic antagonists, antimuscarinic drugs, tricyclic antidepressants, SNRIs, beta adrenoceptor agonists, phosphodiesterase inhibitors, potassium channel openers, nociceptin/orphanin FQ (OP4) agonists, neurokinin (NK1 and NK2) antagonists, P2X3 antagonists, musculotrophic drugs and sacral neuromodulation. Specific agents include oxybutinin, emepronium, tolterodine, flavoxate, flurbiprofen, tolterodine, dicyclomine, propiverine, propantheline, dicyclomine, imipramine, doxepin, duloxetine and 1-deamino-8-Darginine vasopressin.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

Compounds of formula I can be prepared by reacting a compound of formula II with a compound of formula III:

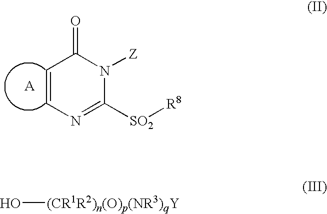

wherein A, R¹, R², R³, Y, Z, n, p and q are as defined above and R⁸ is a C₁₋₆alkyl group such as methyl. The reaction is generally carried out in the presence of an amine such as NH₃ or NEt₃ at a temperature from about 20 to 60° C.

Compounds of formula II can be prepared by reacting a compound of formula (I):

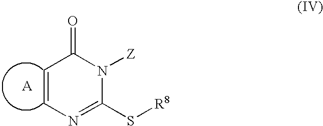

wherein A, R⁸ and Z are as defined above with an oxidising agent such as m-chloroperbenzoic acid (MCPBA), generally in a solvent such as dichloromethane (DCM) at about room temperature.

Compounds of formula IV can be prepared by reacting a compound of formula V with a compound of formula VI:

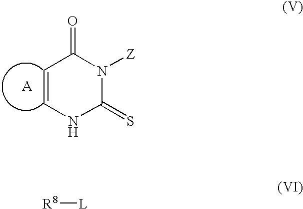

wherein A, R⁸ and Z are as defined above and L is a leaving group such as halogen, particularly iodine, generally in the presence of a base such as potassium carbonate and a solvent such as acetonitrile (MeCN) at about room temperature.

Compounds of formula V can be prepared by reacting a compound of formula VII:

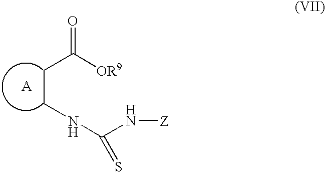

wherein A and Z are as defined above and R⁹ is a C₁₋₆alkyl group such as ethyl, with a base such as potassium hydroxide or sodium hydroxide, generally in a solvent such as water at a temperature from about 50 to 80° C.

Compounds of formula VII can be prepared by reacting a compound of formula VIII with a compound of formula IX:

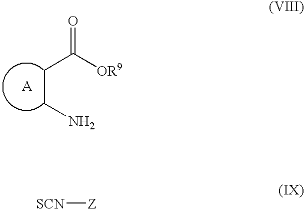

wherein A, R⁹ and Z are as defined above. The reaction is generally carried out in a solvent such as pyridine at about 45° C.

In an alternative process, compounds of formula I can be prepared by reacting a compound of formula X with a compound of formula III:

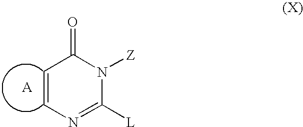

wherein A and Z are as defined above and L is a leaving group such as chlorine. The reaction is generally carried out in the presence of a base such as potassium carbonate and in a solvent such as acetonitrile (MeCN) at a temperature from about 50° to 80° C.

Compounds of formula X wherein L is chlorine can be prepared by reacting a compound of formula V with a chlorinating agent such as POCl₃ at about 135° C.

Where the synthesis of intermediates and starting materials is not described these compounds are commercially available or can be made from commercially available compounds by standard methods, or by extension from the Descriptions and Examples herein.

Compounds of formula I may be converted to other compounds of formula I by known methods or by methods described in the Descriptions and Examples.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### DESCRIPTION 1

### 1-(4-Chlorolphenyl)-9-methyl-2-thioxo-1,2,3,9-tetrahydro-6H-purin-6-one hydrochloride

To a solution of ethyl 3-nitriloalaninate (Synthesis, 1996, 11, 1325; 20 g, 0.156 mol) in MeCN (400 ml) was added triethylorthoformate (26 ml, 23.2 g, 0.156 mol) and the resulting solution heated to 90° C. After 1 h the solution was cooled to room temperature and a solution of methylamine (8 M in ethanol, 20 ml, 0.156 mol) added and the reaction stirred at RT for 18 h. The reaction was condensed in vacuo to a viscous oil then taken up in hydrochloric acid (1 N, 180 ml). The aqueous layer was washed with dichloromethane (2×200 ml, 1×100 ml). The aqueous layer was neutralised by the addition of solid sodium bicarbonate (˜20 g) and then extracted with dichloromethane (5×200 ml). The organic layers were combined, dried over MgSO₄ and condensed in vacuo to give a brown/red solid residue. The residue was slurried in ethyl acetate (40 ml) with sonication, filtered, then the solid rinsed with ether and dried to give ethyl 5-amino-1-methyl-1H-imidazole-4-carboxylate (9.88 g, 37%). Ethyl 5-amino-1-methyl-1H-imidazole-4-carboxylate (4.5 g, 26.6 mmol) and 4-chlorophenyl isothiocyanate (4.5 g, 26.6 mmol) were stirred in pyridine (22 ml) at 45° C. for 18 h. The suspension was cooled and diluted by the addition of ice. When the ice had melted the reaction was filtered, the product rinsed with water and diethyl ether to give ethyl 5-({[(4-chlorophenyl)amino]carbonothioyl}amino)-1-methyl-1H-imidazole-4-carboxylate. The solid was slurried in 1% aqueous sodium hydroxide solution (150 ml) and heated at 80° C. for 30 mins. The reaction was filtered to remove insoluble impurities and then acidified to pH˜5 using hydrochloric acid (5 N), causing a thick white suspension to form. The mixture was aged for 30 minutes and filtered. The solid was rinsed with water then diethyl ether and dried to give the title compound as a white solid (5.28 g, 68%). ¹H NMR (360 MHz, DMSO) δ 7.58 (1H, s), 7.37 (2H, m), 7.06 (1H, br. s), 6.96 (2H, m), 3.54 (3H, s). M/z (ES⁺) 293, 295 (M+H⁺).

### DESCRIPTION 2

### 1-(4-Chlorophenyl)-9-ethyl-2-thioxo-1,2,3,9-tetrahydro-6H-purin-6-one hydrochloride

To a solution of ethyl 3-nitriloalaninate (Synthesis, 1996, 11, 1325; 25 g, 0.195 mol) in MeCN (400 ml) was added triethylorthoformate (32.5 ml, 28.9 g, 0.195 mol) and the resulting solution heated to 90° C. After 70 min the solution was cooled to room temperature and a solution of ethylamine (2 M in tetrahydrofuran, 98 ml, 0.195 mol) added and the reaction stirred at RT for 18 h. The reaction was condensed in vacuo to a viscous oil then taken up in hydrochloric acid (1 N, 200 ml). The aqueous layer was washed with dichloromethane (2×200 ml, 1×100 ml). The aqueous layer was neutralised by the addition of solid sodium bicarbonate (˜25 g) and then extracted with dichloromethane (5×200 ml). The organic layers were combined, dried over MgSO₄ and condensed in vacuo to give a brown/red solid residue. The residue was slurried in ethyl acetate (50 ml), filtered, and the solid rinsed with diethyl ether and dried to give ethyl 5-amino-1-ethyl-1H-imidazole-4-carboxylate (13.0 g, 36%). Ethyl 5-amino-1-ethyl-1H-imidazole-4-carboxylate (0.62 g, 3.4 mmol) and 4-chlorophenyl isothiocyanate (0.58 g, 3.4 mmol) were stirred in pyridine (2 ml) at 45° C. for 18 h. The suspension was cooled and diluted by the addition of ice. When the ice had melted the reaction was extracted into ethyl acetate. The organic layer was dried over MgSO₄ and evaporated in vacuo to give a mixture of the symmetrical thiourea N,N′-bis(4-chlorophenyl)thiourea and ethyl 5-{[(4-chlorophenyl)amino]carbonothioyl}amino)-1-ethyl-1H-imidazole-4-carboxylate (1.12 g). The solid was slurried in 1% aqueous sodium hydroxide solution (20 ml) and heated at 90° C. for 16 h. The reaction was filtered, the filtrate evaporated in vacuo, and the residue was diluted with methanol and loaded onto a strong cation exchange (SCX) cartridge. The cartridge was washed with methanol and dichloromethane and then the product eluted with 2 M methanolic ammonia. After drying, this gave the title compound (756 mg, 72%). ¹H NMR (400 MHz, CD₃OD) δ 7.76 (1H, s), 7.43 (2H, d, J 8.5), 7.13 (2H, d, J 8.7), 4.16 (2H, q, J 7.3), 1.45 (3H, t, J 7.2). M/z (ES⁺) 307, 309 (M+H⁺).

### DESCRIPTION 3

### 2-Chloro-1-(4-chlorophenyl)-9-ethyl-1,9-dihydro-6H-purin-6-one

Description 2 (860 mg, 2.5 mmol) was suspended in a large excess of phosphorous oxychloride (>20 eq) and dissolved upon heating to 135° C. The solution was then heated at this temperature for a further 36 h. The reaction mixture was cooled, evaporated in vacuo, and azeotroped twice with toluene. The resulting sticky brown oil was dissolved in dichloromethane then neutralised with sat. NaHCO₃ (aq). The dichloromethane layer was dry loaded onto a silica flash column, eluting product with ethyl acetate/dichloromethane (1:1) to give the title compound as a pale yellow solid (426 mg, 55%). ¹H NMR (500 MHz, CDCl₃) δ 7.79 (1H, s), 7.52 (2H, d, J 8.6), 7.21 (2H, d, J 8.6), 4.23 (2H, q, J 7.3), 1.56 (3H, t, J 7.3). M/z (ES⁺) 309, 311 (M+H⁺).

### DESCRIPTION 4

### 2-Chloro-1-(4-chlorophenyl)-9-methyl-1,9-dihydro-6H-purin-6-one

Prepared from Description 1 according to the procedure outlined in Description 3. ¹H NMR (360 MHz, DMSO) δ 8.14 (1H, s), 7.64 (2H, d, J 8.6), 7.52 (2H, d, J 8.6), 3.76 (3H, s). M/z (ES⁺) 295, 297 (M+H⁺).

### DESCRIPTION 5

### 1-(4-Fluorophenyl)-9-methyl-2-thioxo-1,2,3,9-tetrahydro-6H-purin-6-one hydrochloride

Prepared from ethyl 3-nitriloalaninate, methylamine and 4-fluorophenyl isothiocyanate according to the procedure described in Description 1. ¹H NMR (360 MHz, DMSO) δ 7.85 (1H, s), 7.31-7.21 (4H, m), 3.76 (3H, s).

### DESCRIPTION 6

### 2-Chloro-1-(4-fluorolphenyl)-9-methyl-1,9-dihydro-6H-purin-6-one

Prepared from Description 5 according to the procedure outlined in Description 3. ¹H NMR (CDCl₃) δ 7.75 (1H, s), 7.23-7.21 (4H, m), 3.83 (3H, s). M/z (ES⁺) 279, 281 (M+H⁺).

### DESCRIPTION 7

### 1-(4-Chlorophenyl)-2-thioxo-9-(2,2,2-trifluoroethyl)-1,2,3,9-tetrahydro-6H-purin-6-one

Prepared from ethyl 3-nitriloalaninate, trifluoroethylamine and 4-chlorophenyl isothiocyanate according to the procedure described in Description 1. ¹H NMR (360 MHz, DMSO) δ 5.30 (2H, q, J 8.7), 7.28 (2H, d, J 8.6), 7.53 (2H, d, J 8.6), 7.99 (1H, s). M/z (ES⁺) 361, 363 (M+H⁺).

### DESCRIPTION 8

### 2-Chloro-1-(4-chlorophenyl)-9-(2,2,2-trifluoroethyl)-1,9-dihydro-6H-Purin-6-one

Prepared from Description 7 according to the procedure outlined in Description 3.

¹H NMR (500 MHz, DMSO) δ 5.23 (2H, q, J 9.0), 7.56 (2H, d, J 8.6), 7.64 (2H, d, J 8.6), 8.25 (1H, s). M/z (ES⁺) 363, 365 (M+H⁺).

### DESCRIPTION 9

### 1-(4-Chlorophenyl)-9-cyclopropyl-2-thioxo-1,2,3,9-tetrahydro-6H-purin-6-one hydrochloride

To a solution of ethyl 3-nitriloalaninate (Synthesis, 1996, 11, 1325; 27 g, 0.21 mol) in MeCN (500 mL) was added triethylorthoformate (35 ml, 31.2 g, 0.25 mol) and the resulting solution heated to 90° C. After 90 min the yellow-green solution was cooled to room temperature and a solution of cyclopropyl amine (17.3 ml, 14.2 g, 0.25 mol) in EtOH (100 ml) was added, causing the solution to go orange. The reaction was stirred at 45° C. for 90 minutes then at room temperature overnight. The reaction was condensed to a viscous oil then taken up in dichloromethane (˜200 ml) and washed with sodium hydroxide solution (2M, 50 ml) then water (50 ml). The aqueous layers were combined and extracted with dichloromethane (2×100 ml). All the organic layers were combined, dried over MgSO₄ and condensed in vacuo to give a brown solid residue. The residue was slurried in minimum EtOH, filtered and the solid rinsed with ether and dried to give ethyl 5-amino-1-cyclopropyl-1H-imidazole-4-carboxylate as a beige solid (6.45 g, 16%). The filtrate also contained product. Ethyl 5-amino-1-cyclopropyl-1H-imidazole-4-carboxylate (4.0 g, 20.5 mmol) and 4-chlorophenyl isothiocyanate (3.47 g, 20.5 mmol) were stirred in pyridine (17 ml) at 45° C. for 24 h. The suspension was cooled and diluted by the addition of ice. When the ice had melted the reaction was filtered, the product rinsed with water and dried to give ethyl 5-(4-chlorophenyl aminocarbonothioylamino)-1-cyclopropyl-1H-imidazole-4-carboxylate (5.68 g). The solid was slurried in 1% aqueous sodium hydroxide solution (25 ml) and heated at 80° C. for 2 h. The reaction was filtered to remove insoluble impurities and then acidified to pH˜5 using hydrochloric acid (5 N), causing a thick white suspension to form. The mixture was aged for 30 minutes, diluted with water and filtered. The solid was rinsed with water then ether and dried to give the title compound as a beige solid (3.95 g, 61%). ¹H NMR (360 MHz, DMSO) δ 7.88 (1H, s), 7.52 (2H, J 8.6), 7.22 (2H, J 8.6), 3.47-3.45 (1H, m), 1.08 (4H, d, J 6.9). M/z (ES⁺) 319, 321 (M+H⁺).

### DESCRIPTION 10

### 1-(4-Chlorophenyl)-9-cyclopropyl-2-(methylthio) 1,9-dihydro-6H-purin-6-one

A mixture of Description 9 (1.0 g, 2.99 mmol), K₂CO₃ (2.0 g, 14.5 mmol) and iodomethane (0.31 ml, 5.03 mmol) in MeCN (30 ml) was stirred at room temperature. After 24 h the reaction was diluted with water and extracted into dichloromethane. The organic layer was dried over MgSO₄ and condensed to give the title compound (1.00 g, 100%). ¹H NMR (500 MHz, CDCl₃) δ 7.68 (1H, s), 7.50 (2H, d, J 8.6), 7.22 (2H, d, J 8.6), 3.43-3.39 (1H, m), 2.53 (3H, s), 1.16 (4H, d, J 7.3). M/z (ES⁺) 333, 335 (M+H⁺).

### DESCRIPTION 11

### 1-(4-Chlorophenyl)-9-cyclopropyl-2-(methylsulfonyl)-1,9-dihydro-6H-purin-6-one

Description 10 (200 mg 0.60 mmol) was added to a solution of m-chloroperbenzoic acid (313 mg, 1.81 mmol) in dichloromethane (10 ml) and the reaction stirred at room temperature. After 30 min molecular sieves (3 Å, ˜1 g of beads) were added. After a further 90 min additional MCPBA (105 mg, 0.60 mmol) was added. After a total of 6 h the reaction was filtered through celite and condensed. The crude reaction mixture was purified by flash column chromatography, eluting with 20 to 25% MeCN in dichloromethane, to give the title sulfone (153 mg, 70%). ¹H NMR (500 MHz, CDCl₃) δ 7.92 (1H, s), 7.50 (2H, d, J 8.7), 7.31 (2H, d, J 8.6), 3.49-3.47 (1H, m), 3.44 (3H, s), 1.26-1.22 (2H, m), 1.22-1.16 (2H, m). M/z (ES⁺) 365, 367 (M+H⁺).

### DESCRIPTION 12

### 4-(9-Methyl-6-oxo-2-thioxo-2,3,6,9-tetrahydro-1H-purin-1-yl)benzonitrile hydrochloride

Prepared from ethyl 3-nitriloalaninate, methylamine and 4-cyanophenyl isothiocyanate, according to the procedure described in Description 1. ¹H NMR (500 MHz, DMSO) δ 7.96 (2H, d, J 8.4), 7.88 (1H, s), 7.46 (2H, d, J 8.4), 3.77 (3H, s). M/z (ES⁺) 284 (M+H⁺).

### DESCRIPTION 13

### 4-[9-Methyl-2-methylthio)-6-oxo-6,9-dihydro-1H-purin-1-yl]benzonitrile

Prepared from Description 12 and iodomethane according to the procedure in Description 10. ¹H NMR (400 MHz, DMSO) δ 8.07 (2H, d, J 8.5), 8.03 (1H, s), 7.68 (2H, d, J 8.5), 3.77 (3H, s), 2.52 (3H, s).

### DESCRIPTION 14

### 4-[9-Methyl-2-(methylsulfonyl)-6-oxo-6,9-dihydro-1H-purin-1-yl]benzonitrile

Prepared from Description 13 according to the procedure in Description 11. ¹H NMR (500 MHz, DMSO) δ 8.34 (1H, s), 8.02 (2H, d, J 8.5), 7.67 (2H, d, J 8.5), 3.86 (3H, s), 3.53 (3H, s). M/z (ES⁺) 330 (M+H⁺).

### DESCRIPTION 15

### Ethyl 2-trifluoromethyl-1,3-thiazole-4-carboxylate

A solution of 2,2,2-trifluoroacetamide (7.12 g, 63 mmol) and Lawesson's Reagent (15.3 g, 37.8 mmol) in THF (60 ml) was stirred at reflux for 18 h. The reaction mixture was cooled, ethyl bromopyruvate (8 ml, 63 mmol) added and the reaction refluxed for 18 h. The reaction was cooled, evaporated in vacuo, and the resulting crude material extracted into ethyl acetate and washed with water. The organic fraction was dried over MgSO₄ and condensed to give a yellow/orange oil. The oil was purified by flash column chromatography on silica eluting with 15% ethyl acetate in hexane to provide the title compound as a clear oil (3 g, 21%). ¹H NMR (400 MHz, CDCl₃) δ 8.39 (1H, s), 4.47 (2H, q, J 7.1), 1.42 (3H, t, J 7.2).

### DESCRIPTION 16

### [2-(Trifluoromethyl)-1,3-thiazol-4-yl]methanol

Di-isobutyl aluminium hydride (1 M in dichloromethane, 25.2 ml, 25.2 mmol) was added dropwise to a solution of Description 15 (2.83 g, 12.6 mmol) in THF (40 ml) at −78° C. The reaction was allowed to stir at −78° C. for 1 h then a further equivalent of diisobutyl aluminium hydride (1 M in dichloromethane, 12 ml, 12 mmol) was added and the solution stirred at −78° C. for another hour. Methanol (20 ml) was added and the solution allowed to warm to room temperature. The reaction was evaporated in vacuo, extracted into diethyl ether, washed with aqueous sodium potassium tartrate (200 ml), then aqueous ammonium chloride (200 ml). The diethyl ether layer was dried over MgSO₄, evaporated in vacuo to give the crude alcohol product as a light yellow oil (2.9 g). ¹H NMR (400 MHz, CDCl₃) δ 7.49 (1H, s), 4.85 (2H, s). M/z (ES⁺) 184 (M+H⁺).

### DESCRIPTION 17

### 2-[4-(Trifluoromethyl)phenyl]ethanol

A solution of Borane in tetrahydrofuran (1.0 M, 14.6 ml, 14.6 mmol) was added slowly to a solution of [4-(trifluoromethyl)phenyl]acetic acid (2.5 g, 11.3 mmol) in tetrahydrofuran (15 ml) in a water bath at room temperature. The reaction was stirred at room temperature for 16 h. The reaction was quenched by the addition of sodium hydroxide solution (2 M, 25 ml) and stirred for 1 h. The reaction was extracted with ethyl acetate (2×100 ml) and the organic layers dried over MgSO₄ and condensed to give the title compound which was used without further purification (2.2 g, quant). ¹H NMR (360 MHz, CDCl₃) a 7.57 (2H, d, J 8.0), 7.35 (2H, d, J 7.9), 3.90 (2H, t, J 6.5), 2.93 (2H, t, J 6.5).

### DESCRIPTION 18

### [4-Trifluoromethyl-1,3-thiazol-2-yl]methanol

A solution of 3-bromo-1,1,1-trifluoroacetone (3.2 ml, 30.2 mmol) and 2-(tert-butylcarbonyloxy) thioacetamide (5.3 g, 30.2 mmol) in ethanol (15 ml) was stirred at reflux for 18 h. To the cooled solution were added methanol (10 ml) and DBU (4.6 ml, 30.2 mmol) and the solution was stirred at room temperature for 2 days. The reaction mixture was evaporated in vacuo, extracted with dichloromethane, washed with water, dried over Na₂SO₄, and evaporated in vacuo to give crude product as a black oil. Purification by flash column chromatography, eluting with 30% ethyl acetate in hexane, gave the title compound as an off-white solid (3.2 g, 58%). ¹H NMR (400 MHz, DMSO) δ 8.42 (1H, s), 6.30 (1H, t, J 5.8), 4.79 (2H, d, J 5.7).

### EXAMPLE 1

### 1-(4-Chlorophenyl)-9-cyclopropyl-2-methoxy-1,9-dihydro-6H-purin-6-one

Description 11 (27 mg, 0.074 mmol) was taken up in a solution of methanolic ammonia (2 M, 2 ml) and the reaction was stirred at 60° C. After 20 min the reaction was condensed and loaded on to a strong cation exchange (SCX) cartridge with methanol. The crude product was eluted with methanolic ammonia (2 M) and condensed. The product was isolated by preparative thin layer chromatography, eluting with 10% methanol in dichloromethane, to give the title compound (15 mg, 64%). ¹H NMR (400 MHz, CDCl₃) δ 7.63 (1H, s), 7.46 (2H, d, J 8.6), 7.15 (2H, d, J 8.6), 3.97 (3H, s), 3.39-3.35 (1H, m), 1.18-1.10 (4H, m). M/z (ES⁺) 317, 319 (M+H⁺).

### EXAMPLE 2

### 4-[9-Methyl-6-oxo-2-(3,3,3-trifluoropropoxy)-6,9-dihydro-1H-purin-1-yl]benzonitrile

Description 14 (45 mg, 0.137 mmol) was heated in a mixture of triethylamine (20 mL) and 3,3,3-trifluoropropanoyl (200 μL) at 60° C. After 15 min excess 3,3,3-trifluoropropanoyl was allowed to evaporate and the reaction was purified by flash column chromatography, eluting with 5% methanol in dichloromethane. The product was purified further using preparative thin layer chromatography, eluting with ethyl acetate/dichloromethane/methanol [50:45:5] to give the title compound (19 mg, 38%). ¹H NMR (400 MHz, CDCl₃) δ 7.79 (2H, d, J 8.5), 7.65 (1H, s), 7.34 (2H, d, J 8.5), 4.61 (2H, t, J 6.1), 3.77 (3H, s), 2.53-2.43 (2H, m). M/z (ES⁺) 364 (M+H⁺).

### EXAMPLE 3

### 1-(4-Chlorophenyl)-9-ethyl-2-(3,3,3-trifluoropropoxy)-1,9-dihydro-6H-purin-6-one

A mixture of Description 3 (100 mg, 0.325 mmol), 3,3,3-trifluoropropanol (74 mg, 0.649 mmol) and K₂CO₃ (134 mg, 0.974 mmol) in MeCN (3 mL) was stirred at 50° C. for 16 h. Additional 3,3,3-trifluoropropanol (30 mg) was added, the temperature raised to 60° C. and the reaction stirred for another 24 h. The reaction was condensed and partitioned between water and dichloromethane. The organic layer was dried over MgSO₄ and evaporated. The crude product was purified firstly by preparative thin layer chromatography, eluting with 10% methanol in dichloromethane and then by flash column chromatography, eluting with ethyl acetate/dichloromethane [1:1] then ethyl acetate/dichloromethane/methanol [50:47.5:2.5] to give the title compound (43 mg, 34%). ¹H NMR (400 MHz, CDCl₃) δ 7.67 (1H, s), 7.45 (2H, d, J 8.6), 7.14 (2H, d, J 8.7), 4.59 (2H, t, J 6.2), 4.16 (2H, q, J 7.3), 2.53-2.45 (2H, m), 1.53 (3H, m). M/z (ES⁺) 387, 389 (M+H⁺).

Examples 4-13 in the table below were made from Descriptions 3, 4, 6 or 8 and the appropriate alcohol according to the procedure described in Example 3. The alcohols used are commercially available or prepared as outlined in Descriptions 15-18.

| | | M/z (ES⁺) | |
|---|---|---|---|
| EX | NAME | [M + H]⁺ | ¹H NMR |
| 4 | 1-(4-Chlorophenyl)-9-methyl-2-{[2- | 442, 444 | (400 MHz, DMSO) δ 7.99 |
| | (trifluoromethyl)-1,3-thiazol-4- | | (1H, s), 7.95 (1H, s), 7.57- |
| | yl]methoxy}-1,9-dihydro-6H-purin-6- | | 7.55 (2H, d, J 6.6), 7.39-7.37 |
| | one | | (2H, d, J 6.6), 5.59 (2H, s), |
| | | | 3.72 (3H, s). |
| 5 | 1-(4-Chlorophenyl)-9-methyl-2-{[4- | 442, 444 | (360 MHz, CDCl₃) δ 7.75 |
| | (trifluoromethyl)-1,3-thiazol-2- | | (1H, s), 7.65 (1 H, s), 7.49 |
| | yl]methoxy}-1,9-dihydro-6H-purin-6- | | (2H, d, J 8.6), 7.21 (2H, d, J |
| | one | | 8.6), 5.71 (2H, s), 3.77 (3H, |
| | | | s). |
| 6 | 1-(4-Fluorophenyl)-9-methyl-2-(3,3,3- | 357 | (500 MHz, CDCl₃) δ 7.63 |
| | trifluoropropoxy)-1,9-dihydro-6H- | | (1H, s), 7.16 (4H, d, J 6.5 Hz), |
| | purin-6-one | | 4.59 (H, t, J 6.2 Hz), 3.75 |
| | | | (3H, s), 2.53-2.45 (2H, m). |
| 7 | 1-(4-Chlorophenyl)-9-methyl-2-(3,3,3- | 373, 375 | (500 MHz, CDCl₃) δ 7.64 |
| | trifluoropropoxy)-1,9-dihydro-6H- | | (1H, s), 7.45 (2H, d, J 8.6), |
| | purin-6-one | | 7.13 (2H, d, J 8.6), 4.60 (2H, |
| | | | t, J 6.2), 3.75 (3H, s), 2.53- |
| | | | 2.45 (2H, m). |
| 8 | 1-(4-Chlorophenyl)-9-methyl-2-{2-[4- | 449, 451 | (500 MHz, CDCl₃) δ 7.62 |
| | (trifluoromethyl)phenyl]ethoxy}-1,9- | | (1H, s), 7.47 (2H, d, J 8.0), |
| | dihydro-6H-purin-6-one | | 7.41 (2H, d, J 8.6), 7.01 (4H, |
| | | | t, J 4.3), 4.56 (2H, t, J 6.3), |
| | | | 3.73 (3H, s), 2.96 (2H, t, J |
| | | | 6.2). |
| 9 | 1-(4-Chlorophenyl)-9-(2,2,2- | 441, 443 | (500 MHz, CDCl₃) δ 7.73 |
| | trifluoroethyl)-2-(3,3,3- | | (1H, s), 7.47 (2H, d, J 8.6), |
| | trifluoropropoxy)-1,9-dihydro-6H- | | 7.14 (2H, t, J 4.3), 4.69 (2H, |
| | purin-6-one | | q, J 8.4), 4.60 (2H, t, J 6.1), |
| | | | 2.53-2.45 (2H, m). |
| 10 | 1-(4-Fluorophenyl)-9-methyl-2-(4,4,4- | 371 | (500 MHz, CDCl₃) δ 7.63 |
| | trifluorobutoxy)-1,9-dihydro-6H- | | (1H, s), 7.18 (4H, d, J 6.5), 4.42 |
| | purin-6-one | | (2H, t, J 6.0), 3.74 (3H, s), |
| | | | 2.02-1.88 (4H, m). |
| 11 | 1-(4-Chlorophenyl)-9-methyl-2-(4,4,4- | 387, 389 | (500 MHz, CDCl₃) δ 7.62 |
| | trifluorobutoxy)-1,9-dihydro-6H- | | (1H, s), 7.48-7.46 (2H, m), |
| | purin-6-one | | 7.15-7.13 (2H, m), 4.43 (2H, t, |
| | | | J 6.1), 3.74 (3H, s), 2.03-1.88 |
| | | | (4H, m). |
| 12 | 1-(4-Fluorophenyl)-9-methyl-2-{[4- | 426 | (360 MHz, DMSO) δ 8.51 |
| | (trifluoromethyl)-1,3-thiazol-2- | | (1H, s), 7.99 (1H, s), 7.40- |
| | yl]methoxy}-1,9-dihydro-6H-purin-6- | | 7.42 (2H, m), 7.39-7.36 (2H, |
| | one | | m), 5.73 (2H, s), 3.74 (3H, s). |
| 13 | 1-(4-Fluorophenyl)-9-methyl-2-{[2- | 426 | ¹H NMR (360 MHz, DMSO) |
| | (trifluoromethyl)-1,3-thiazol-4- | | δ 7.95 (1H, s), 7.94 (1H, s), |
| | yl]methoxy}-1,9-dihydro-6H-purin-6- | | 7.39-7.37 (2H, m), 7.34-7.32 |
| | one | | (2H, m), 5.59 (2H, s), 3.72 |
| | | | (3H, s). |

The above exemplified compounds of the present invention have been tested in the following assay and generally possess an IC₅₀<300 nM and, in the majority of cases, <200 nM. Other assays, such as electrophysiology using rat VR1 expressed in HEK cells measuring activity at various pH levels, can be used.

### Determination of In Vitro Activity

In vitro activity of compounds was measured using one or both of the following assays.

### Method 1

CHO cells, stably expressing recombinant rat or human VR1 receptors and plated into black-sided 384-well plates, were washed three times with assay buffer (containing Hepes, NaCl₂, KCl, MgCl₂, CaCl₂, sucrose, glucose and probenecid, pH 7.4) and then incubated with test compound and 4 uM Fluo-3-AM for 60 minutes at room temperature in darkness. Cells were washed three times more to remove excess dye, before being placed, along with plates containing capsaicin and test compounds into a Molecular Devices FLIPR³⁸⁴. The FLIPR³⁸⁴ simultaneously performed automated pharmacological additions and recorded fluorescence emission from Fluo-3. In all experiments, basal fluorescence was recorded, before re-addition of test compounds and subsequent addition of a previously determined concentration of capsaicin that evoked 80% of the maximum response. Inhibition of capsaicin evoked increases in intracellular [Ca²⁺] were expressed relative to wells on the same plate to which an EC80 concentration of capsaicin was added in the absence of test compounds.

### Method 2

Antagonists were ranked by absolute efficacy at a single low concentration vs. activation by either pH 5.5 or capsaicin (500 nM) using a medium-throughput electrophysiology assay. TRPV1 activity is initially determined using a 5 second application of 500 nM capsaicin. Agonist (either pH 5.5 or capsaicin) is then applied for 5 seconds followed by a 30 second wash period until a stable control response is achieved. Inhibition of the agonist response is determined following applications of a single concentration of test compound and inhibition is monitored using repeated agonist activation in the presence of the compound until a stable inhibition state is achieved (up to a maximum of 10 minutes of application). If a successful recovery was achieved by re-applying a control wash, additional compounds can be tested sequentially. Inhibition effect of the drug is calculated as the sustained maximum current within the 5 second agonist application divided by the control sustained maximum current before the drug had been applied, multiplied by 100 (=% inhibition@ the test concentration).

1. A compound of formula I: 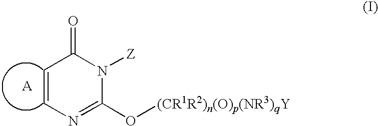
wherein:
A is a fused five-membered heteroaromatic ring containing 1, 2 or 3 heteroatoms independently chosen from O, N and S, providing that no more than one O or S atom is present, or a fused six-membered heteroaromatic ring containing 1, 2 or 3 N atoms;
A is optionally substituted by one, two or three groups independently chosen from halogen, hydroxy, S(O)ᵣC₁₋₄alkyl, S(O)ᵣNR⁴R⁵, —NR^{X}S(O)ᵣC₁₋₄alkyl, formyl, C₁₋₄alkylcarbonyl, C₁₋₆alkyl, haloC₁₋₆alkyl, hydroxyC₁₋₆alkyl, C₁₋₆alkoxy, haloC₁₋₆alkoxy, hydroxyC₁₋₆alkoxy, C₃₋₇cycloalkyl, C₃₋₇cycloalkoxy, C₂₋₆alkenyl, C₂₋₆alkynyl, amino, nitro, cyano, C₁₋₆alkylamino, di(C₁₋₆alkyl)amino, aminoC₁₋₆alkyl, aminoC₁₋₆alkoxy, C₁₋₆alkylaminoC₁₋₆alkyl, di(C₁₋₄alkyl)aminoC₁₋₆alkyl, C₁₋₆alkylaminoC₁₋₆alkoxy, di(C₁₋₆alkyl)aminoC₁₋₆alkoxy, and a phenyl, naphthyl, a five-membered heteroaromatic ring containing one, two, three or four heteroatoms independently chosen from O, N or S, at most one heteroatom being O or S, and a six-membered heteroaromatic ring containing one, two or three N atoms, the ring being optionally substituted by halogen, hydroxy, cyano, nitro, NR⁴R⁵ as defined below, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
R¹ and R² are independently hydrogen, hydroxy, halogen or C₁₋₆alkyl;
R³ is hydrogen or C₁₋₆alkyl;
n is zero, one, two, three or four;
p is zero or one;
when p is zero, q is zero or one;
when p is one, q is zero;
r is zero, one or two;
Y is hydrogen, C₁₋₆alkoxy, haloC₁₋₆alkoxy, C₁₋₆alkyl, haloC₁₋₆alkyl, hydroxyC₁₋₆alkyl, aminoC₁₋₆alkyl, carboxyC₁₋₆alkyl, C₃₋₇cycloalkyl, haloC₃₋₇cycloalkyl; or a phenyl ring, a five-membered heteroaromatic ring containing one, two, three or four heteroatoms independently chosen from O, N and S, providing that no more than one O or S atom is present, a six-membered heteroaromatic ring containing one, two or three N atoms, or a nine- or ten-membered fused bicyclic heteroaromatic ring containing a phenyl ring or a six-membered heteroaromatic ring as just defined, fused to either a six-membered heteroaromatic ring as just defined or a five-membered heteroaromatic ring as just defined, a six-membered saturated ring containing one or two heteroatoms independently chosen from O and N, the ring being optionally substituted by one or more groups independently selected from halogen, C₁₋₆alkyl, C₂₋₆alkenyl, C₂₋₆alkynyl, nitro, cyano, C₃₋₇cycloalkyl, hydroxy, C₁₋₆alkoxy, haloC₁₋₆alkyl, haloC₁₋₆alkoxy, hydroxyC₁₋₆alkyl, hydroxyC₁₋₆alkoxy, phenyl, an unsubstituted five-membered heteroaromatic ring as just described, a six-membered heteroaromatic ring as just described, a six-membered saturated ring as just described and NR⁴R⁵;
each R⁴ and R⁵ is independently hydrogen or C₁₋₆alkyl or R⁴ and R⁵, together with the nitrogen atom to which they are attached, may form a saturated 4-7 membered ring;
R^{X} is hydrogen or C₁₋₆alkyl;
Z is a phenyl ring, a five-membered heteroaromatic ring containing one, two, three or four heteroatoms independently chosen from O, N or S, at most one heteroatom being O or S, or a six-membered heteroaromatic ring containing one, two or three N atoms, optionally substituted by halogen, hydroxy, cyano, nitro, NR⁴R⁵ as defined above, S(O)ᵣNR⁴R⁵, —NR^{X}S(O)ᵣC₁₋₄alkyl, S(O)ᵣC₁₋₄alkyl, C₁₋₆alkyl, C₂₋₆alkenyl, C₂₋₆alkynyl, haloC₁₋₆alkyl, C₁₋₆alkoxy, haloC₁₋₆alkoxy, C₃₋₇cycloalkyl or hydroxyC₁₋₆alkyl;
provided that when n is zero then p and q are both zero and Y is not hydrogen, C₁₋₆alkoxy or haloC₁₋₆alkoxy;
provided that when A is thiophene, n is one, p and q are zero, R¹ and R² are hydrogen and Z is 4-chlorophenyl then Y is not 3-fluorophenyl;
or a pharmaceutically acceptable salt or tautomer thereof.

2. A compound of claim 1 of formula IA: 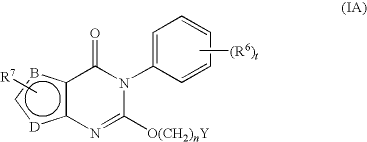
wherein:
n and Y are as defined in claim 1;
t is zero, one or two;
one of B and D is N and the other N or S;
R⁶ is cyano, halogen, C₁₋₄alkyl, haloC₁₋₄alkyl, C₁₋₄alkoxy, haloC₁₋₄alkoxy, amino, C₁₋₄alkylamino or di(C₁₋₆)alkylamino; and
R⁷ is hydrogen, halogen, hydroxy, C₃₋₅cycloalkyl, C₁₋₄alkyl, haloC₁₋₄alkyl, C₁₋₄alkoxy or haloC₁₋₄alkoxy;
provided that when n is zero then Y is not hydrogen, C₁₋₆alkoxy or haloC₁₋₆alkoxy;
or a pharmaceutically acceptable salt or tautomer thereof.

3. A compound of claim 2 of formula IB: 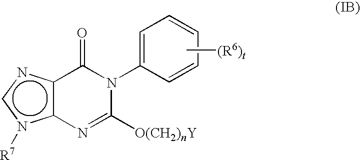
wherein:
n, t, R⁶, R⁷ and Y are as defined in claim 2;
or a pharmaceutically acceptable salt or tautomer thereof.

4. A compound selected from:
1-(4-chlorophenyl)-9-cyclopropyl-2-methoxy-1,9-dihydro-6H-purin-6-one;
4-[9-methyl-6-oxo-2-(3,3,3-trifluoropropoxy)-6,9-dihydro-1H-purin-1-yl]benzonitrile;
1-(4-chlorophenyl)-9-ethyl-2-(3,3,3-trifluoropropoxy)-1,9-dihydro-6H-purin-6-one;
1-(4-chlorophenyl)-9-methyl-2-{[2-(trifluoromethyl)-1,3-thiazol-4-yl]methoxy}-1,9-dihydro-6H-purin-6-one;
1-(4-chlorophenyl)-9-methyl-2-{[4-(trifluoromethyl)-1,3-thiazol-2-yl]methoxy}-1,9-dihydro-6H-purin-6-one;
1-(4-fluorophenyl)-9-methyl-2-(3,3,3-trifluoropropoxy)-1,9-dihydro-6H-purin-6-one;
1-(4-chlorophenyl)-9-methyl-2-(3,3,3-trifluoropropoxy)-1,9-dihydro-6H-purin-6-one;
1-(4-chlorophenyl)-9-methyl-2-{2-[4-(trifluoromethyl)phenyl]ethoxy}-1,9-dihydro-6H-purin-6-one;
1-(4-chlorophenyl)-9-(2,2,2-trifluoroethyl)-2-(3,3,3-trifluoropropoxy)-1,9-dihydro-6H-purin-6-one;
1-(4-fluorophenyl)-9-methyl-2-(4,4,4-trifluorobutoxy)-1,9-dihydro-6H-purin-6-one;
1-(4-chlorophenyl)-9-methyl-2-(4,4,4-trifluorobutoxy)-1,9-dihydro-6H-purin-6-one;
1-(4-fluorophenyl)-9-methyl-2-{[4-(trifluoromethyl)-1,3-thiazol-2-yl]methoxy}-1,9-dihydro-6H-purin-6-one;
1-(4-fluorophenyl)-9-methyl-2-{[2-(trifluoromethyl)-1,3-thiazol-4-yl]methoxy}-1,9-dihydro-6H-purin-6-one;
and pharmaceutically acceptable salts and tautomers thereof.

5. A pharmaceutical composition comprising, a compound of claim 1 or pharmaceutically acceptable salts thereof in association with a pharmaceutically acceptable carrier or excipient.

6. (canceled)

7. (canceled)

8. (canceled)

9. A process for the preparation of a compound of formula I as defined in claim 1, which comprises:
(A) reacting a compound of formula II with a compound of formula III: 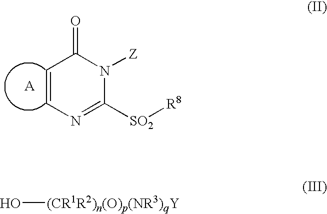
wherein A, R¹, R², R³, Y, Z, n, p and q are as defined in claim 1 and R⁸ is a C₁₋₆alkyl group; or
(B) reacting a compound of formula X with a compound of formula III: 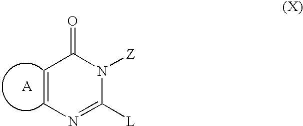
wherein A and Z are as defined in claim 1 and L is a leaving group.

10. A method for the treatment or prevention of physiological disorders that may be ameliorated by modulating VR1 activity, which method comprises administration to a patient in need thereof of an effective amount of a compound of claim 1 or a composition comprising a compound of claim 1.

11. A method for the treatment or prevention of a disease or condition in which pain and/or inflammation predominates, which method comprises administration to a patient in need thereof of an effective amount of a compound of claim 1, or a composition comprising a compound of claim 1.
